(19) United States
(12) Patent Application Publication
(10) Pub. No.: US 2022/0203022 A1
(43) Pub. Date: Jun. 30, 2022

(54) ON-BODY DRUG DELIVERY DEVICE WITH ANTENNAS SECURED OUTSIDE OF A HOUSING FOR WIRELESS COMMUNICATIONS

(71) Applicant: Insulet Corporation, Acton, MA (US)

(72) Inventors: David NAZZARO, Groveland, MA (US); John D'ARCO, Wilmington, MA (US); Nicholas CONTE, Harvard, MA (US); Kepei SUN, Andover, MA (US)

(21) Appl. No.: 17/553,699

(22) Filed: Dec. 16, 2021

Related U.S. Application Data
(60) Provisional application No. 63/131,865, filed on Dec. 30, 2020.

(51) Int. Cl. A61M 5/142; G16H 40/67; G16H 20/17

(52) U.S. Cl. CPC A61M 5/14248; G16H 40/67; A61M 2205/3584; A61M 2205/3553; G16H 20/17

(57) ABSTRACT

An on-body drug delivery device may have one or more antennas secured outside a housing of the on-body drug delivery device for facilitating wireless communications. The one or more antennas may be secured to an adhesive pad that adheres the on-body drug delivery device to a user. Alternatively, the one or more antennas may be secured to a portion of the housing for the on-body drug delivery device. In such instances, the one or more antennas may pass through one or more holes in the housing to facilitate an electrical connection with a component, like a wireless communications transceiver, inside the housing. Alternatively, wireless communications with the one or more antennas and the component may be used.

### RELATED APPLICATIONS

This application claims the benefit of U.S. Provisional Patent Application No. 63/131,865, filed Dec. 30, 2020, the contents of which are incorporated herein by reference in their entirety.

### BACKGROUND

Some conventional on-body drug delivery devices provide a wireless communication capability. For instance, some conventional on-body drug delivery devices may communicate with another device via the Bluetooth® wireless protocol. In order to participate in such wireless communications, a conventional on-body drug delivery device needs an antenna. The antenna is used to wirelessly transmit and receive signals. Such conventional on-body drug delivery devices implement the antenna as a trace antenna on a printed circuit board (PCB). In other words, the antenna is realized as metal trace deposited on a top surface of a PCB inside a housing of a conventional on-body drug delivery device.

Unfortunately, there are drawbacks to using such a trace antenna on a PCB in an on-body drug delivery device. First, the trace antenna occupies valuable surface area on the PCB. The surface area occupied by the trace antenna could be used for other components, such as sensors or other electronics. Alternatively, the size of the PCB and perhaps, as a result, the size of the on-body drug delivery device could be reduced but for the space occupied by the trace antenna. Second, the efficiency of the trace antenna is low due to absorption by the body of the user and due to interference in signal reception and transmission attributable to other electronic components on the PCB.

### SUMMARY

In accordance with an inventive aspect, an on-body drug delivery device includes a housing and a wireless communications transceiver positioned inside the housing for transmitting and receiving wireless communications. The on-body drug delivery device also includes at least one antenna positioned outside of the housing for wireless communication. The at least one antenna has a communication connection with the wireless communications transceiver.

The device may include an adhesive pad for securing the on-body drug delivery device to the user, and the at least one antenna may be secured to the adhesive pad. The antenna may be, for example, laminated with the adhesive pad, woven into material of the adhesive pad or secured to the adhesive pad via adhesive. In other embodiments, the at least one antenna is positioned on an outer surface of the housing. In some embodiments, the antenna may be printed on material that forms the adhesive pad. In some embodiments, multiple antennas forming an antenna array may be printed on the adhesive pad or on an outer surface of the housing. The communication connection may be a wired connection that extends through the housing. Alternatively, the communication connection may be a wireless connection. The at least one antenna may include an antenna array. A digital signal processor (DSP) may be included in the device, and the DSP may be configured to perform beamforming with the antenna array.

In accordance with another inventive aspect, an insulin delivery device may include an adhesive pad for securing the insulin delivery device to the user. The insulin delivery device may include a wireless communications transceiver for transmitting and receiving wireless communications. Further, the insulin delivery device may include at least one antenna secured to the adhesive pad and having a communication connection with the wireless communications transceiver.

In some instances, multiple antennas may be secured to the adhesive pad. The insulin delivery device may include a beamformer for beamforming with the antennas. The wireless communications transceiver may communicate in accordance with a Bluetooth protocol, a Bluetooth Low Energy protocol, a WiFi protocol, or another wireless protocol. The at least one antenna may be laminated with the adhesive pad, woven into material of the adhesive pad or secured to the adhesive pad via adhesive.

In accordance with another inventive aspect, a method includes encapsulating a wireless communications transceiver for transmitting and receiving wireless communications inside a housing of an on-body drug delivery device. At least one antenna may be secured outside of the housing for wireless communication on behalf of the on-body drug delivery device and/or may be secured to the adhesive pad. In some embodiments, multiple antennas forming an antenna array may be secured outside of the housing and/or to the adhesive pad. A communication connection is created between the wireless communications transceiver and the at least one antenna. The method may include securing an adhesive pad to the on-body drug delivery device for securing the on-body drug delivery device to a user.

### BRIEF DESCRIPTION OF THE DRAWINGS

FIG. 1 depicts a block diagram of an illustrative on-body drug delivery system for an exemplary embodiment.

FIG. 2 depicts an illustrative on-body drug delivery device for an exemplary embodiment with an antenna secured to an adhesive pad for the on-body drug delivery device.

FIG. 3 depicts an illustrative on-body drug delivery device for an exemplary embodiment with multiple antennas secured to an adhesive pad for the on-body drug delivery device.

FIG. 4A depicts a side view of an illustrative on-body drug delivery device for an exemplary embodiment where multiple antennas are secured to a top surface of an adhesive pad and pass through the housing of the on-body drug delivery device.

FIG. 4B depicts a side view of an illustrative on-body drug delivery device for an exemplary embodiment where multiple antennas are secured in an adhesive pad and pass through the housing of the on-body drug delivery device.

FIG. 4C depicts a side view of an illustrative on-body drug delivery device for an exemplary embodiment where multiple antennas are secured in an intermediate layer of an adhesive pad and pass through the housing of the on-body drug delivery device.

FIG. 5 depicts a view of a top surface of an illustrative on-body drug delivery device housing with antennas secured thereto in an exemplary embodiment.

FIG. 6 depicts a flowchart of illustrative steps that may be performed in an exemplary embodiment to establish a communication connection between a wireless transceiver and antenna(s).

FIG. 7 depicts a diagram of illustrative wireless communication protocols that may be used in an exemplary embodiment.

FIG. 8 depicts a flowchart of illustrative steps that may be performed in an exemplary embodiment to perform beamforming.

### DETAILED DESCRIPTION

Exemplary embodiments may provide an on-body drug delivery device with one or more antennas secured outside a housing of the on-body drug delivery device. The one or more antennas may be secured to an adhesive pad that adheres the on-body drug delivery device to a user. For example, the one or more antennas may be adhered to the adhesive pad, printed on the material forming the adhesive pad, laminated between layers of the adhesive pad or otherwise secured to the adhesive pad. In such instances, the one or more antennas may pass through one or more holes in the housing to facilitate an electrical connection with a component, such as a wireless communications transceiver, inside the housing.

The exemplary embodiments avoid the problem encountered with trace antennas in conventional on-body drug delivery devices of using valuable surface area on a PCB inside the housing for the trace antenna. Since, in exemplary embodiments, the one or more antennas are secured outside of the housing and not on a PCB inside the housing, no valuable surface area is used for the one or more antennas. Thus, the size of the PCB may be shrunk and potentially the size of the on-body drug delivery device may be reduced. Alternatively, the surface area on the PCB that is not occupied by the one or more antennas may be used by other electronic components, such as sensors, hence, increasing the capabilities and/or reducing the size of the on-body drug delivery device.

The exemplary embodiments also may improve the transmission and reception relative to trace antennas in conventional on-body drug delivery devices. By being positioned outside of the housing of the on-body drug delivery device, the one or more antennas of the exemplary embodiments does not have to contend with interference from other electronic components on the PCB of the on-body drug delivery device. In exemplary embodiments where the one or more antennas are secured to the adhesive patch of the on-body drug delivery device, the high dielectric properties of the human body may be exploited to reduce the size of the one or more antennas. In addition, the exemplary embodiments may deploy multiple antennas on a single on-body drug delivery device, and as a result, beamforming may be used to boost radiation gain to certain directions. This ability to direct the radiation energy is helpful for communicating with an off-body device. Because the antennas use the high dielectric properties of the body of the user, the antennas may be small in size and hence, may form antenna arrays.

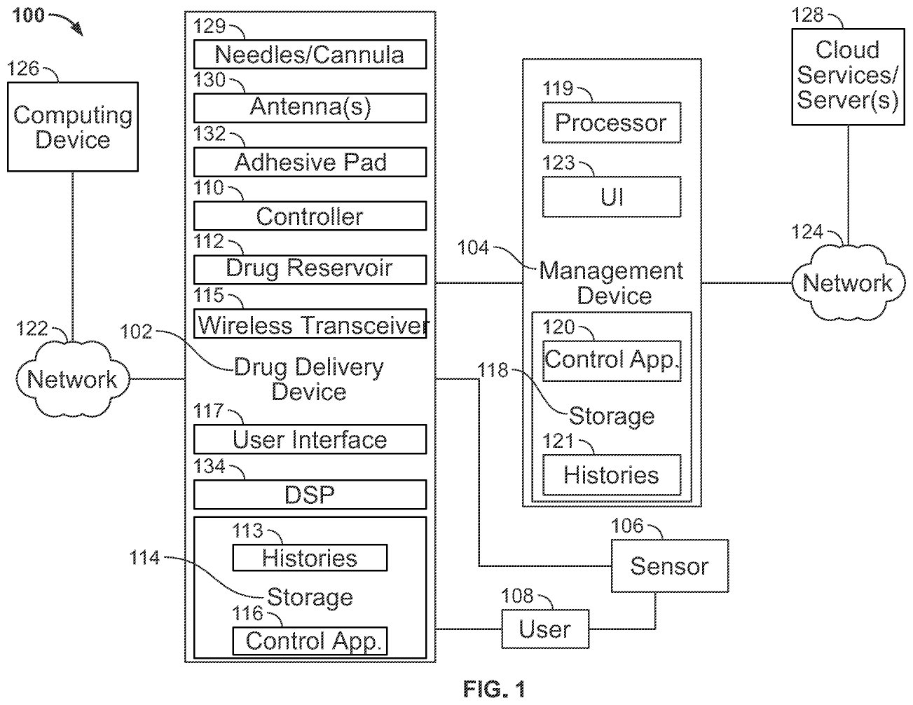

FIG. 1 depicts an illustrative on-body drug delivery system (100) suitable for an exemplary embodiment that includes an on-body drug delivery device (102) as the on-body medical device. The on-body drug delivery device (102) may be directly coupled to a user (e.g., directly attached to a body part and/or skin of the user (108) via an adhesive or the like). In an example, the on-body drug delivery device (102) may include a cannula and/or needles (129) for insertion into tissue of a user (108) for delivery of a drug. The on-body drug delivery device (102) may include an adhesive pad (132) to facilitate attachment to the user (108). The adhesive pad (132) may be thermally welded to the underside of the housing of the on-body drug delivery device (102), secured to the housing via an adhesive or otherwise secured to the housing. The adhesive pad (132) includes an adhesive surface for securing to the skin of the user (108) to help hold the on-body drug delivery device (102) to the user (108).

The on-body drug delivery device (102) may include a controller (110). The controller (110) may be implemented in hardware, software, or any combination thereof. The controller (110) may be, for example, a microprocessor, a logic circuit, a field programmable gate array (FPGA), an application specific integrated circuit (ASIC) or a microcontroller coupled to a memory. The controller (110) may maintain a date and time as well as other functions (e.g., calculations or the like). The controller (110) may be operable to execute a control application (116) stored in the storage (114) that enables the controller (110) to direct operation of the on-body drug delivery device (102). The storage (114) may hold histories (113) for a user. Where the on-body drug delivery device (102) is an insulin delivery device, the histories (113) may include information such as a history of automated insulin deliveries, a history of bolus insulin deliveries, meal event history, exercise event history, and the like. In addition, the controller (110) may be operable to receive data, instructions, or information. The storage (114) may include both primary memory and secondary memory. The storage (114) may include random access memory (RAM), read only memory (ROM), optical storage, magnetic storage, removable storage media, solid state storage or the like.

The on-body drug delivery device (102) may include a drug reservoir (112) for storing a drug, such as insulin, therapeutics, painkillers, chemotherapy agents, glucagon, hormonal agents, blood thinners, antibiotics, antidepressants, anti-anxiety agents, antipsychotics, birth control agents, statins, blood pressure control agents or the like, for delivery to the user (108). A fluid path to the user (108) may be provided, and the on-body drug delivery device (102) may expel the drug from the drug reservoir (112) to deliver the drug to the user (108) via the fluid path. The fluid path may, for example, include tubing coupling the on-body drug delivery device (102) to the user (108) (e.g., tubing coupling a needles/cannula (129) to the drug reservoir (112)).

There may be one or more wireless communications links with one or more devices physically separated from the on-body drug delivery device (102) including, for example, a management device (104) of the user and/or a caregiver of the user and an analyte sensor (106). The one or more antennas (130) facilitate transmission and reception of such wireless communications with other devices. A wireless transceiver (115) may be provided to transmit and receive wireless communications via the one or more antennas (130). The communication links may include wireless communication links operating according to any known communications protocol or standard, such as the Bluetooth® standard, the Bluetooth® Low Energy (BLE) standard, Wi-Fi (IEEE 802.11), a cellular standard, or any other wireless protocol or standard. The on-body drug delivery device (102) may also include a user interface (117), such as an integrated display device for displaying information to the user (108) and in some embodiments, receiving information from the user (108). The user interface (117) may include a touchscreen and/or one or more input devices, such as buttons, knob or a keyboard. A digital signal processor (DSP) (134) may be provided to perform digital signal processing, including acting as a beamformer when multiple antennas (130) are used.

The on-body drug delivery device (102) may interface with a network (122). The network (122) may include a local area network (LAN), a wide area network (WAN) or a combination therein. A computing device (126) may be interfaced with the network, and the computing device may communicate with the on-body drug delivery device (102).

The drug delivery system (100) may include a sensor (106) for sensing an analyte obtained from the user (108). The analyte being sensed may be blood glucose concentration, lactate levels, ketone levels, sodium levels, potassium levels, uric acid levels, alcohol levels or the like. The sensor (106) may in some exemplary embodiments provide periodic blood glucose concentration measurements and may be a continuous glucose monitor (CGM), or another type of device or sensor that provides blood glucose measurements. The sensor (106) may be physically separate from the on-body drug delivery device (102) or may be an integrated component thereof. The sensor (106) may be coupled to the user (108) by, for example, adhesive or the like and may provide information or data on one or more medical conditions and/or physical attributes of the user (108). The information or data provided by the sensor (106) may be used to adjust drug delivery operations of the on-body drug delivery device (102).

The drug delivery system (100) may also include management device (104). In some exemplary embodiments, there is no management device (104) or the management device (104) may be optional or may only be used to activate drug delivery device (102). Instead, the functionality of the management device (104) is incorporated in the on-body drug delivery device (102). The management device (104) may be a special purpose device, such as a dedicated personal diabetes manager (PDM) device. Alternatively, the management device (104) may be a programmed general-purpose device, such as any portable electronic device including, for example, a dedicated controller, such as a processor, a smartphone, a smartwatch or a tablet. The management device (104) may be used to activate or program or adjust operation of the on-body drug delivery device (102) and/or the sensor (106). The management device (104) may be any portable electronic device including, for example, a dedicated controller, a smartphone, a smartwatch, or a tablet. In the depicted example, the management device (104) may include a processor (119) and a storage (118). The processor (119) may execute processes to manage a user's blood glucose levels and for controlling the delivery of the drug or therapeutic agent to the user (108). The processor (119) may also be operable to execute programming code stored in the storage (118). For example, the storage (118) may be operable to store one or more control applications (120) for execution by the processor (119). The storage (118) may store the control application (120), histories (121) like those described above for the on-body drug delivery device (102) and other data and/or programs. In another example, after activation, the on-body drug delivery device (102) may operate without the management device (104) by communicating directly with the sensor (106) and delivering a drug based on those communications using an automated drug delivery (ADD) application or control application (116) stored in memory on the on-body drug delivery device (102).

The management device (104) may include a user interface (UI) (123) for communicating with the user (108). The user interface (123) may include a display, such as a touchscreen, for displaying information. The touchscreen may also be used to receive input when it is a touched. The user interface (123) may also include input elements, such as a keyboard, button, knobs or the like.

The management device (104) may interface with a network (124), such as a LAN or WAN or combination of such networks. The management device (104) may communicate over network (124) with one or more servers or cloud services (128).

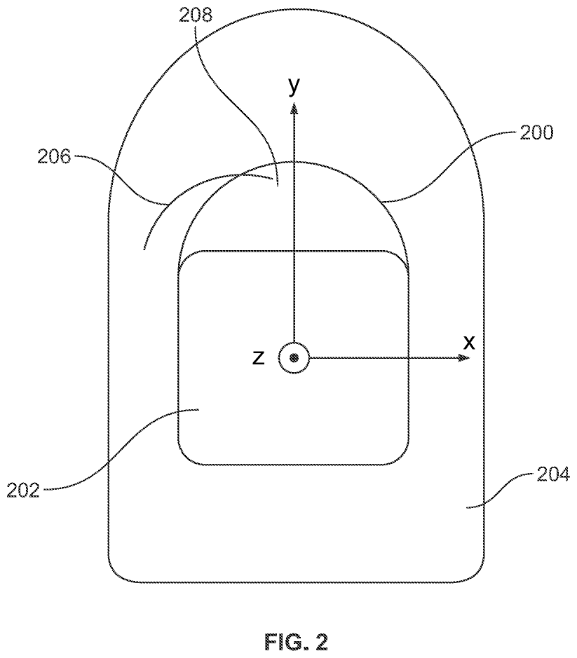

FIG. 2 depicts a top view of the on-body drug delivery device (200) of an exemplary embodiment. The on-body drug delivery device (200) includes at least one housing that encases components, such as the controller (110), drug reservoir (112), storage (114), wireless transceiver (115) and DSP (134). Adhesive pad (204) is secured to the bottom of the at least one housing (202) via thermal welding, adhesive or other securing mechanism. The bottom surface of the adhesive pad (204) is coated with an adhesive for adhering the adhesive pad (204) with the skin of the user (108), such as on the abdomen, lower back or arm of the user (108). A single antenna (206) is shown in the embodiment of FIG. 2. The antenna (206) is secured to the adhesive pad (204). The antenna (206) should be flexible to conform to the skin surface of the user (108). The antenna (206) may be a metal trace made of a metal such as copper, aluminum or nickel. A hole (208) may be provided in the at least one housing (202). The antenna (206) may pass through the hole (208) into the interior of the at least one housing (202) and electrically connected with the wireless transceiver (115). The antenna (206) may be used to transmit and receive wireless communications.

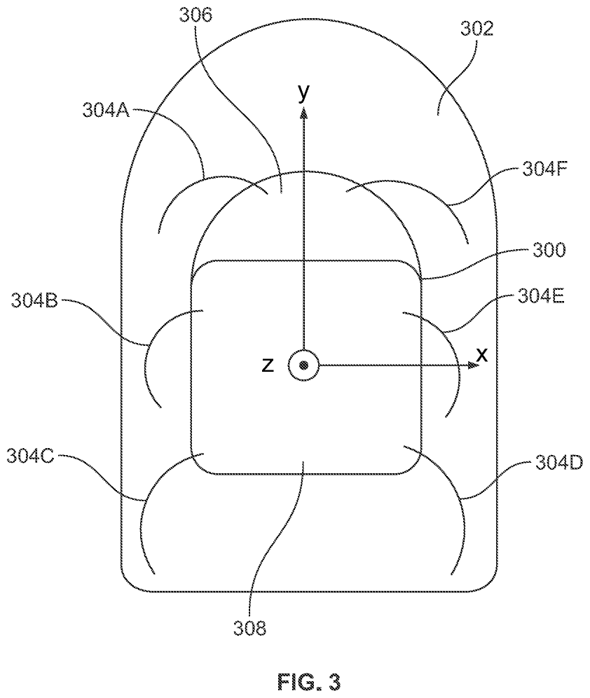

FIG. 3 depicts a top view of an alternative exemplary embodiment of an on-body drug delivery device (300). The on-body drug delivery device (300) of FIG. 3 differs from the on-body drug delivery device (200) of FIG. 2 in that it has multiple antennas (304A, 304B, 304C, 304D, 304E and 304F) organized in an array rather than a single antenna (206). The on-body drug delivery device (300) includes an adhesive pad (302). Holes, like hole (306), are provided for each antenna (304A-304F) to pass to the interior and to be electrically connected with the DSP (134) and wireless transceiver (115). As was mentioned above, with the multiple antennas (304A-304F) of the antenna array, radiation energy may be directed in desired directions. Beamforming may be performed using the DSP (134). The DSP (134) is configured to perform such beamforming. This may be useful in communicating with other medical devices by directing the formed beams to the other medical devices. The beamforming avoids problems, such as the wireless communications being absorbed by the body of the user and transmitted energy dispersing and being directed away from the intended target. The multiple antennas (304A-304F) also helps in obtaining better reception of wireless communications from certain directions, such as from the directions of the sensor (106) and the management device (104).

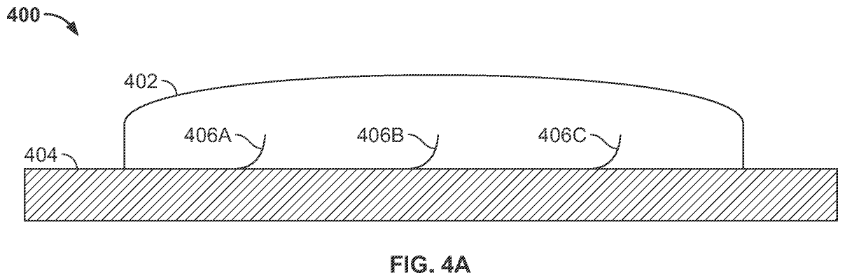

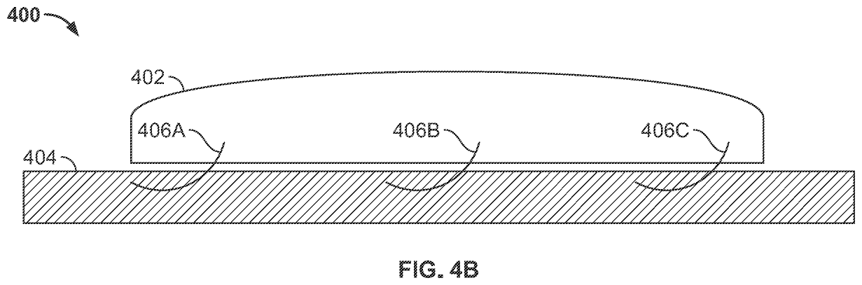

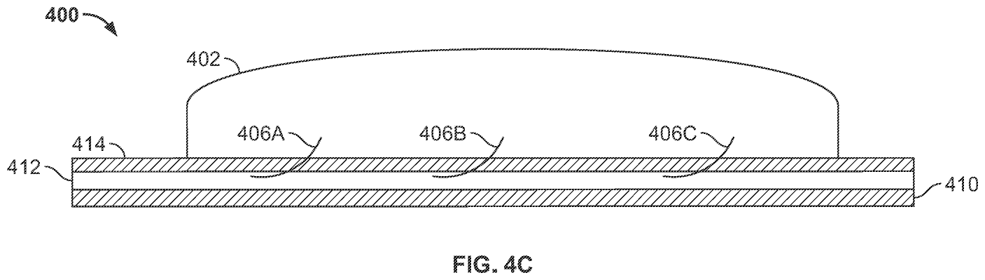

The antenna(s) (130) may be secured to the adhesive pad (130) in a number of different ways. FIG. 4A depicts a side view of the on-body drug delivery device (400) that shows a first way. Antennas (406A, 406B and 406C) are secured to a top surface of adhesive pad (404). The antennas (406A, 406B and 406C) may be secured by an adhesive, by being deposited on the top surface of the adhesive pad (404) or by being printed on material that forms the adhesive pad (404) or by being otherwise secured to the top surface of the adhesive pad (404). As can be seen, the antennas (406A, 406B and 406C) pass into the interior of the at least one housing (402) for electrical connection with a component, such as the wireless transceiver (115) and/or the DSP (134).

FIG. 4B depicts an example of another way of securing the antennas (406A, 406B and 406C) to the adhesive pad (404). In this example, the antennas (406A, 406B and 406C) are inserted into the adhesive pad (404). The antennas (406A, 406B and 406C) may be inserted by being woven into the fabric or substrate of the adhesive pad (404). Alternatively, the antennas (406A, 406B and 406C) may be inserted into the adhesive pad (404) after formation of the adhesive pad (404). The antennas (406A, 406B and 406C) pass into the interior of the at least one housing (402), as has been described above.

FIG. 4C depicts a third way of securing the antennas (406A, 406B and 406C) to the adhesive pad (404). In this example, the antennas (406A, 406B and 406C) are laminated between layers (410) and (414) of the adhesive pad (404). The antennas (406A, 406B and 406C) are embedded in an intermediate layer (412), such as by being woven into a substrate. In some exemplary embodiments, the antennas (406A, 406B and 406C) themselves are laminated between layers (410) and (414) and not embedded in a separate substrate. The antennas (406A, 406B and 406C) extend through layer (414) into the interior of the housing (402) of the on-body drug delivery device (400).

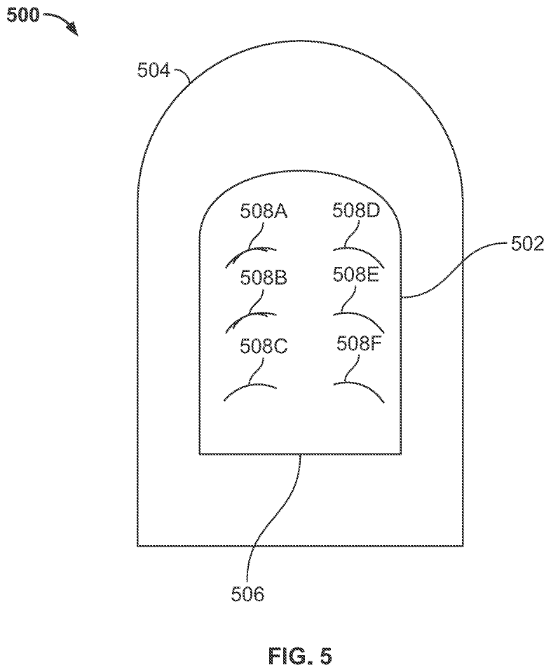

FIG. 5 depicts another alternative for the on-body drug delivery device (500). In this alternative, the antennas (508A, 508B, 508C, 508D and 508E) are formed on a top surface of the at least one housing (502) and are not secured to the adhesive pad (504). The antennas (508A, 508B, 508C, 508D and 508E) may be metallic traces that are secured to the top surface of the at least one housing (504) by an epoxy or adhesive, for example. Still further, the metallic traces for the antennas (508A, 508B, 508C, 508D and 508E) may be deposited or printed onto the top surface of the at least one housing (504).

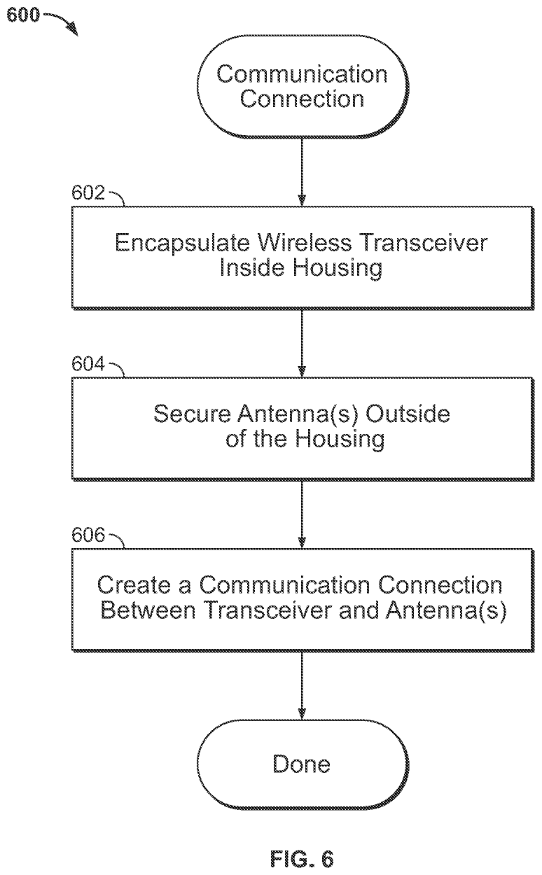

In order for the antenna(s) (130) to be used for wireless communications, a communication connection must be established between the wireless transceiver (115) and the antenna(s) (130). FIG. 6 provides a flowchart (600) of illustrative steps that may be performed to realize the communication connection. The wireless communications transceiver (115) is encapsulated inside the housing of the on-body drug delivery device (102) (602). The wireless communication transceiver (115) may, for example, reside on a PCB inside the housing. Antenna(s) is/are secured outside of the at least one housing (604), such as on the top surface of the at least one housing, or on the adhesive pad, such as described above. A communication connection is then established between the wireless communication transceiver (115) and the antenna(s) (130) (606). The communications connection may be a wired connection, such as by way of an electrical connection or may be a wireless connection.

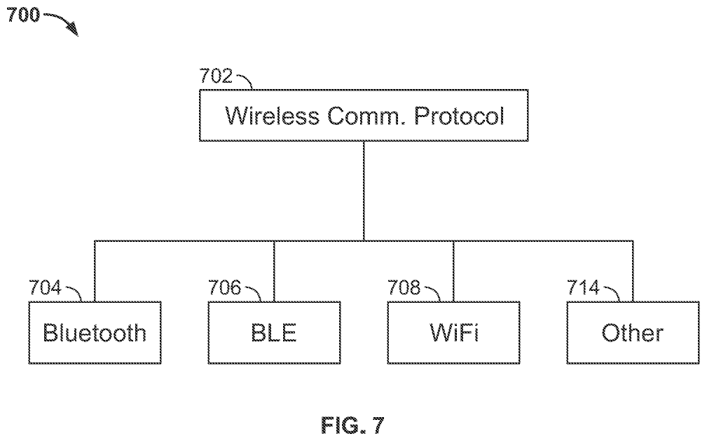

A number of different wireless communication protocols (702) may be used to communicate via the antenna(s) (130), such as shown in the diagram (700) of FIG. 7. The wireless protocol may be a Bluetooth® protocol (704) or a Bluetooth® Low Energy (BLE) protocol (706). The wireless protocol may be a WiFi (IEEE 802.11) protocol (708). Other wireless protocols (714) may also be used.

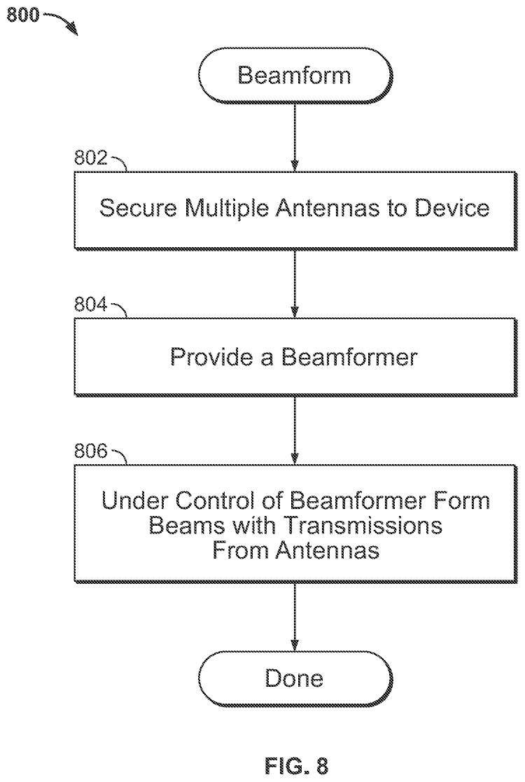

As was mentioned above, the antennas may be used to perform beamforming with the help of the DSP (134). FIG. 8 depicts a flowchart (800) of illustrative steps that may be performed to realize such beamforming. Initially, multiple antennas (130) forming an antenna array are secured to the on-body drug delivery device (102), such as described above. A beamformer is provided (804). The DSP (134) may act as the beamformer to output one or more beams via the antennas (130). Other hardware may be used to perform the needed digital signal processing. Under the control of the beamformer, beams are formed and transmitted via the antennas (130) of the antenna array (806).

The exemplary embodiments thus enable a medical device, like an on-body medical device such as an insulin pump, to have one or more antennas that may be used for wireless communications. Since the antennas are located outside the housing of the medical device, the antennas do not occupy valuable space inside the housing, such as on a PCB, and may enable the medical device to be smaller. In addition, the positioning of the antenna(s) outside of the housing of the medical device avoids interference with communications due to other components positioned in close proximity to the antenna(s). In one embodiment, the antenna(s) are positioned on an adhesive pad that helps secure the medical device to a user. In that embodiment, the large dielectric properties of the body of the user, allows the antenna(s) size(s) to be small. Hence, multiple antennas in an antenna array may be secured to the medical device. With the antenna array, beamforming may be used to enhance the quality of wireless communications.

While exemplary embodiments have been described herein, various changes in form and detail may be made to the exemplary embodiments while still be encompassed by the claims as appended hereto.

1. An on-body drug delivery device, comprising:
at least one housing;
a wireless communications transceiver positioned inside the at least one housing for transmitting and receiving wireless communications; and
at least one antenna positioned outside of the at least one housing for wireless communication, said at least one antenna having a communication connection with the wireless communications transceiver.

2. The on-body drug delivery device of claim 1, further comprising an adhesive pad for securing the on-body drug delivery device to the user and wherein the at least one antenna is secured to the adhesive pad.

3. The on-body drug delivery device of claim 2, wherein the at least one antenna is laminated with the adhesive pad, woven into material of the adhesive pad or secured to the adhesive pad via adhesive.

4. The on-body drug delivery device of claim 2, wherein the at least one antenna is printed on material that forms the adhesive pad.

5. The on-body drug delivery device of claim 1, wherein the at least one antenna is positioned on an outer surface of the at least one housing.

6. The on-body drug delivery device of claim 1, wherein the communication connection is a wired connection that extends through the at least one housing.

7. The drug connection of claim 1, wherein the communication connection is a wireless connection.

8. The on-body drug delivery device of claim 1, wherein the at least one antenna comprises an antenna array of multiple antennas.

9. The on-body drug delivery device of claim 8, further comprising an adhesive pad for securing the on-body drug delivery device to the user and wherein the antennas of the antenna array are secured to the adhesive pad.

10. The on-body drug delivery device of claim 1, further comprising a digital signal processor (DSP).

11. The on-body drug delivery device of claim 10, wherein there are multiple antennas forming an antenna array and the DSP is configured to perform beamforming with the antenna array.

12. An insulin delivery device, comprising:
an adhesive pad for securing the insulin delivery device to the user;
a wireless communications transceiver for transmitting and receiving wireless communications; and
at least one antenna secured to the adhesive pad and having a communication connection with the wireless communications transceiver.

13. The insulin delivery device of claim 12, wherein multiple antennas are secured to the adhesive pad.

14. The insulin delivery device of claim 12, further comprising a beamformer for beamforming with the antennas.

15. The insulin delivery device of claim 12, wherein the wireless communications transceiver communicates in accordance with a Bluetooth protocol, a Bluetooth Low Energy protocol, or a WiFi protocol.

16. The insulin delivery device of claim 12, wherein the at least one antenna is laminated with the adhesive pad, woven into material of the adhesive pad or secured to the adhesive pad via adhesive.

17. A method, comprising:
encapsulating inside a housing of an on-body drug delivery device a wireless communications transceiver positioned for transmitting and receiving wireless communications;
securing at least one antenna outside of the housing for wireless communication on behalf of the on-body drug delivery device; and
creating a communication connection between the wireless communications transceiver and the at least one antenna.

18. The method of claim 17, further comprising securing an adhesive pad to the on-body drug delivery device for securing the on-body drug delivery device to a user.

19. The method of claim 18, the securing the at least one antenna comprises securing the at least one antenna to the adhesive pad.

20. The method of claim 19, wherein the securing the at least one antenna comprises securing multiple antennas to the adhesive pad to form an antenna array secured to the adhesive pad.
